(19) United States
(12) Patent Application Publication
(10) Pub. No.: US 2025/0146025 A1
(43) Pub. Date: May 8, 2025

(54) PROCESS FOR WHOLE-CELL BIOSYNTHESIS OF STYRENE

(71) Applicant: SHELL USA, INC., Houston, TX (US)

(72) Inventors: Hanan Latif Fahmi MESSIHA, Manchester (GB); David LEYS, Manchester (GB)

(21) Appl. No.: 18/838,044

(22) PCT Filed: Mar. 2, 2023
(86) PCT No.: PCT/US2023/063547

§ 371 Date: Aug. 13, 2024

(30) Foreign Application Priority Data
Mar. 3, 2022 (EP) 22160036.4

(51) Int. Cl. C12P 5/00; C12N 9/88

(52) U.S. Cl. CPC C12P 5/005; C12N 9/88; C12Y 403/01024

(57) ABSTRACT

A process for producing styrene converts Z-phenylalanine using a first biocatalyst comprising a PAL enzyme (pheny-lalanine ammonia lyase from Rhodorotula glutinis EC 4.3.1.24) and a second biocatalyst comprising a Fdc1 enzyme (ferulic acid de-carboxylase from Aspergillus niger EC 4.1.1.102). The first and second biocatalysts are provided as whole-cell pellets or derivatives thereof. Styrene is produced by converting the L-phenylalanine to trans-cinnamic acid with the first biocatalyst and converting the trans-cinnamic acid to styrene with the second biocatalyst.

### FIELD OF THE INVENTION

The present invention relates to the field of styrene production, and in particular to whole-cell biosynthesis of styrene.

### BACKGROUND OF THE INVENTION

Styrene is a large-volume commodity petrochemical with a diverse range of commercial applications. As a monomer, styrene is extensively used for producing a wide selection of industrial polymer and co-polymers such as polystyrene, acrylonitrile-butadiene-styrene, styrene-acrylonitrile, and styrene-butadiene rubber. Recent applications of polystyrene include its use in the synthesis of nanobeads in the lateral flow assay for SARS-COV-2 antibody detection, and in the polystyrene-toner centrifugal microfluidic device for rapid molecular diagnosis of COVID-19.

Due to the wide applications of styrene polymers and functional co-polymers, there is an increasing demand for styrene production. The global market for styrene was valued at 56.5 billion USD in 2022 and is anticipated to reach 77.92 billion USD by 2028. Conventional production of styrene depends on chemical catalysis, an energetically demanding route that relies mainly on fossil-fuel resources.

In an effort to reduce greenhouse gases and the overall carbon footprint for producing styrene, efforts have been made to produce styrene via a biological route. Fortunately, bio-production of styrene occurs naturally in various organisms such as bacteria, yeast, and certain plant species making bio-production of styrene feasible. The biosynthesis of styrene from the naturally occurring amino acid L-phenylalanine (L-Phe) occurs via trans-cinnamate. Non-oxidative deamination of L-Phe is catalysed by phenylalanine ammonia-lyase (PAL) to trans-cinnamate, which is then followed by a decarboxylation reaction catalysed by ferulic acid decarboxylase (Fdc1) to produce styrene.

Conventional approaches have focussed on producing styrene in microbial hosts, including recombinant hosts such as Escherichia coli (E. coli) and Saccharomyces cerevisiae that have been engineered to produce styrene. For example, McKenna et al. (“Styrene biosynthesis from glucose by engineered E. coli” Metabol Eng 13:544-554; 2011) provide a list of strains, plasmids and oligonucleotide primers used in its study.

Referencing Mckenna et al, among others, Shen et al. (“Recent Advances in Metabolically Engineered Microorganisms for the Production of Aromatic Chemicals Derived from Aromatic Amino Acids” Frontiers in Bioengineering and Biotechnology 3:407; 1-43; May 2020) provide an overview of articles about metabolic engineering microorganisms for producing aromatic compounds, aromatic acid derivatives (including phenylalanine derivatives, tyrosine derivatives, and tryptophan derivatives), stilbenes, and benzylisoquinoline alkaloids.

One major issue that currently hinders high-titre production is styrene toxicity on growing cells; the highest styrene concentration achieved so far in vivo in Escherichia coli is 350 mg/l. Other approaches attempting to improve this titre through genome engineering, in-vitro, cell-free styrene biosynthesis and strain improvement for biomanufacturing of toxic compounds, have improved styrene production to 4.2-5.3 g/L. Nevertheless, these approaches are not yet economically viable, and the challenging task of developing a cost-effective, eco-friendly and renewable bioprocess to meet the commercial demand for styrene remains.

Shen et al. (ibid) suggest that current titres and yields are still too low for industrial production. Shen et al. suggest prospects to improve titres and yields are adaptive laboratory evolution to improve tolerance of host cells, whole-cell biocatalysts, identification of better enzymes, modular co-cultures, chemically inducible chromosomal evolution, and developing microbial strains.

Li et al. (US2020/0232000A1, 23 Jul. 2020) proposed a biocatalytic route (pathway) to produce “natural” 2-PE, PA, PAA, and PEA from biobased L-phenylalanine and glucose and demonstrated the deamination-decarboxylation of L.-phenylalanine by lyase and decarboxylase to produce styrene with up to 15 g/L.

Grubbe et al. (“Cell-free styrene biosynthesis at high titers” Metab Eng 61:89-95; 2020) relates to a cell-free platform for styrene biosynthesis to circumvent toxicity limits of styrene in vivo. Grubbe et al. used cell-free protein synthesis in E. coli crude extracts to independently express phenylalanine ammonia lyase 2 (PAL2) from Arabidopsis thaliana and ferulic acid decarboxylase 1 (FDC1) from Saccharomyces cerevisiae. The extracts were combined with L-Phe and buffer to produce styrene in vitro. Grubbe et al. achieved a maximum styrene titre of 40.33±1.03 mM (4.20±0.11 g/L.). To prepare E. coli cell extracts, cells were pelleted via centrifugation and flash-frozen. Frozen cells were thawed on ice, resuspended, and then lysed. Cell debris was removed, and the supernatant was flash-frozen.

There is a need for an economically viable process for biosynthesis of styrene. Specifically, there remains a need to reduce the operating expenses and capital expenses of product recovery schemes, while providing a quality product.

### SUMMARY OF THE INVENTION

According to one aspect of the present invention, there is provided a process for producing styrene, the process comprising the steps of: providing a source of L-phenylalanine to a vessel; adding a first biocatalyst comprising a PAL enzyme to the vessel, the first biocatalyst provided in a form selected from PAL-containing whole-cell pellets, a suspension of PAL-containing whole-cell pellets, a suspension of PAL-containing whole cells derived from PAL-containing whole-cell pellets, and combinations thereof; adding a second biocatalyst comprising a Fdc1 enzyme to the vessel, the second biocatalyst provided in a form selected from Fdc1-containing whole-cell pellets, a suspension of Fdc1-containing whole-cell pellets, a suspension of Fdc1-containing whole cells derived from whole-cell pellets, and combinations thereof; and producing styrene by converting the L-phenylalanine to trans-cinnamic acid with the first biocatalyst and converting the trans-cinnamic acid to styrene with the second biocatalyst.

### BRIEF DESCRIPTION OF THE DRAWINGS

The process of the present invention will be better understood by referring to the following detailed description of preferred embodiments and the drawings referenced therein, in which:

FIG. 1 is a graphical illustration of results for the Examples presented herein;

FIGS. 2A and 2B are graphical illustrations of results showing stability of lyophilised whole-cell biocatalysts; and

FIG. 3 is a graphical illustration showing results of tests for styrene production at different quantities of lyophilised biocatalysts.

### DETAILED DESCRIPTION OF THE INVENTION

The present invention provides a process for producing styrene. A source of L-phenylalanine (“L-Phe”) is provided to a vessel. A first biocatalyst comprising a PAL (phenylalanine ammonia-lyase) enzyme and a second biocatalyst comprising a Fdc1 (ferulic acid decarboxylase) enzyme are added to the vessel. The first and second biocatalysts are each independently provided in a form selected from whole-cell pellets, a suspension of whole-cell pellets, a suspension of whole cells derived from whole-cell pellets, and combinations thereof. The PAL enzyme catalyses conversion of L-Phe by non-oxidative deamination to trans-cinnamic acid, while the Fde1 enzyme catalyses the conversion of trans-cinnamic acid by decarboxylation to styrene, according to the reaction scheme:

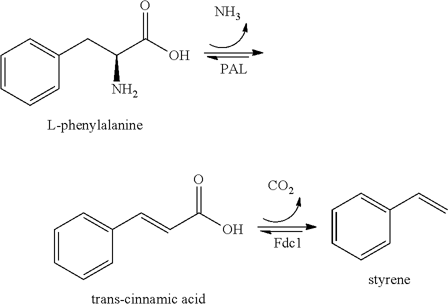

The inventors have surprisingly discovered that by providing the enzymes as whole-cell pellets or derivatives thereof, the conversion to styrene was significantly higher than previously achieved by in-vitro and cell-free conversion of a feedstock to styrene. By providing the Fdc1 and PAL enzymes in a non-growth phase, the process does not suffer from styrene toxicity. Further, by using biocatalysts in a whole-cell form, the cost of separating the enzymes is avoided.

By “whole-cell pellets,” we mean that whole cells comprising the enzyme of interest are harvested from its growth media and pelletized. The whole-cell pellets are in a non-growing phase or condition. The biocatalyst is not harvested from the cells. In one embodiment, the whole-cell pellets are in a resting phase. In a preferred embodiment, the whole cell pellets are lyophilised. Derivatives of whole-cell pellets include a suspension of whole-cell pellets in a carrier liquid, a suspension of whole cells derived from whole-cell pellets, and combinations thereof.

The process of the present invention uses a source of L-Phe. The source of L-Phe may be a fermentation broth, extractant from a protein-rich feedstock, and/or a feedstock that is rich in L-Phe.

In one embodiment, the source of L-Phe is a fermentation broth. In this embodiment, a renewable feedstock is fermented to produce L-Phe. Several microbial strains capable of producing L-Phe from carbon sources have been reported. As non-limiting examples, L-Phe can be produced according to the methods described in Kuvaea et al. (U.S. Pat. No. 9,873,898, 23 Jan. 2018) and Stoynova et al. (U.S. Pat. No. 9,175,319, 3 Nov. 2015), which describe producing L-amino acids using a bacterium of the family Enterobacteriaceae, especially the genus Escherichia. Other examples include, without limitation, E. coli strains, such as, WSH-Z06 (pAP-B03) [35.4 g/L], HD-A2 [62.5 g/L], BR-42 (pAP-B03) [57.6 g/L], x11p3 [61.3 g/1], X11p21 [72.9 g/L], and FUS4.11 pF81kan [13.4 g/L], and Corynebacterium glutamicum strains, including for example, CCRC 18335 strain [23.2 g/L].

The fermentation of bacteria for converting the renewable feedstock to L-Phe may be performed in a manner similar to conventional fermentation methods wherein L-amino acid is produced using a microorganism.

The culture medium for fermentation can be either a synthetic or natural medium. The fermentation medium contains a carbon source, a nitrogen source, a sulphur source, inorganic ions, and other organic and inorganic components, as required. Suitable carbon sources include, without limitation, saccharides such as glucose, lactose, galactose, fructose, arabinose, maltose, xylose, trehalose, ribose, sucrose, and hydrolysates of starches; alcohols such as ethanol, glycerol, mannitol, and sorbitol; organic acids such as gluconic acid, fumaric acid, citric acid, malic acid, and succinic acid; fatty acids; and the like can be used. Examples of nitrogen sources include, without limitation, inorganic ammonium salts such as ammonium sulphate, ammonium chloride, and ammonium phosphate; organic nitrogen such as soybean hydrolysates; ammonia gas; aqueous ammonia; and the like can be used. A suitable sulphur source includes, without limitation, ammonium sulphate, magnesium sulphate, ferrous sulphate, manganese sulphate, and the like.

Preferably, one or more of the carbon, nitrogen and sulphur sources comprises a renewable feedstock derived from animal, vegetable, microbial, and/or bio-derived or mineral-derived waste materials suitable for the production of fuels, fuel components and/or chemical feedstocks.

The culture medium may include a phosphorus source in addition to a carbon source, a nitrogen source, and a sulphur source. Examples of a suitable phosphorus source include, without limitation, potassium dihydrogen phosphate, dipotassium hydrogen phosphate, phosphate polymers such as pyrophosphoric acid. Vitamins such as, without limitation, B1, B2, B6, nicotinic acid, nicotinamide, and B12 may be present in the culture medium as needed, along with organic nutrients such as nucleic acids such as adenine and RNA, or yeast extract.

Fermentation is performed under aerobic conditions at a temperature in a range of from 30 to 45° C., preferably in a range of from 30 to 37° C. and a pH in a range of from about 5 to 8, preferably in a range of from about 6.0 and 7.5. The pH is advantageously adjusted using an inorganic or organic acidic or alkaline substance, and/or ammonia gas.

The fermentation broth may be transferred to another vessel or the first and second biocatalysts may be added to the vessel in which the L-Phe was produced. The fermentation broth may be used with or without a step or removing solids such as cells and cell debris from the liquid medium by centrifugation or membrane filtration, further including recovering L-Phe from the fermentation liquor by concentration, ion-exchange chromatography, and/or crystallization. Advantageously, in this embodiment, the first and second biocatalyst is added to the fermentation broth directly, for example, once a predetermined concentration of L-Phe has been achieved in fermentation, and/or once the fermentation step proceeds to completion. In this way, costs associated with separating L-Phe can be avoided.

In another embodiment, the source of L-Phe is prepared by extraction from a protein- rich renewable feedstock. Extraction is preferred when, for example, the L-Phe is locked in the feedstock.

By “protein-rich” renewable feedstock, we mean a feedstock derived from an animal, vegetable, or microbial source and/or by-product, wherein the feedstock has a protein content of at least 15 wt. % on a dry weight basis, preferably at least 20 wt. % on a dry weight basis, more preferably 90 wt. % on a dry weight basis.

Examples of suitable protein-rich renewable feedstock include, without limitation, dried distillers grain, algae oil, wastes from bioethanol, bio-oil and food production, plant leaves, poultry feather meal, and combinations thereof.

De Schouwer et al. (“Protein-Rich Biomass Waste as a Resource for Future Biorefineries: State of the Art, Challenges, and Opportunities” ChemSusChem 12:1272-1303;2019) describes various processes for extracting amino acids from protein-rich waste streams.

Extraction of L-Phe may be selected from acid hydrolysis, enzymatic hydrolysis, amino acid fractionation, and combinations thereof.

In a further embodiment, the source of L-Phe is a feed rich in L-phenylalanine. Examples of a feed rich in L-Phe include, without limitation, casamino acid, pumpkin seeds, blue green algae, and combinations thereof. The feed in this embodiment has free L-Phe available for enzymatic conversion by the first and second biocatalysts.

The source of the L-Phe is provided to a vessel. In the embodiment, of L-Phe produced by fermentation, the vessel may be the same or different than the fermentation vessel, with or without separation from cells used for producing L-Phe.

The first biocatalyst comprising a PAL (phenylalanine ammonia-lyase) enzyme and the second biocatalyst comprising a Fdc1 (ferulic acid decarboxylase) enzyme are then added to the vessel.

The first and second biocatalysts are each heterologously produced in a microbial host, for example, in an E. coli strain, such as, for example, as E. coli BL21 (DE3) strain. The Fdc1 construct also includes expression of UbiX, the flavin prenyltransferase that yields the prenylated flavin mononucleotide (prFMN) cofactor required for the Fdc1 decarboxylation activity.

A specific example provided herein includes recombinant RgPAL plasmid from Rhodotorula glutinis (in pET16b vector) transformed into E. coli BL21 (DE3) strain for the first biocatalyst. A specific example for the second biocatalyst involves co-expression of Aspergillus niger Fdc1 (AnFdc1) with E. coli (EcUbiX) by cloning both genes into pBbA1C vector. The resulting AnFdc1/EcUbiX in pBbA1C plasmid is transformed and expressed in E. coli BL21 (DE3).

As noted above, in a preferred embodiment, the whole-cell biocatalysts are in a lyophilised condition. Lyophilised cells, which are freeze-dried from fresh cell pellets, can be distributed, and stored at room temperature. This is particularly advantageous for industrial deployments, especially as compared to the storing and handling of fresh cell pellets. Further, lyophilised cells are stable, for example, for up to 9 months and possibly beyond that.

After the growth phase, the whole-cells are harvested by centrifugation to prepare cell pellets. The whole-cell pellets are preferably washed to remove excess culture medium, for example with a saline solution. To maintain the whole-cell biocatalysts in a resting phase, the washed pellets are stored at low temperature, for example in a range from −10° C. to −25° C., preferably at −20° C. To maintain the whole-cell biocatalysts in a lyophilised condition, the washed pellets are frozen in liquid nitrogen and then subjected to freeze-drying. The lyophilised whole-cell pellets may be stored at a temperature in a range from 4° C. to 20° C. The whole-cell pellets are typically in the form of dried powder and/or flakes after lyophilisation.

The first and second biocatalysts may be added to the source of L-Phe at the same time or in any sequence of the first and second biocatalyst. Where there is a delay between adding the first and second biocatalysts, the first biocatalyst preferably precedes addition of the second biocatalyst. When resting whole cells are used, the first and second biocatalysts are added in an amount ranging from 10 to 30 g of cell dry weight (cdw)/L. When lyophilised cells are used, the first and second biocatalyst are added in an amount ranging from 1-10 g/L.

The ratio of first to second biocatalysts are selected based on enzyme activity and expression levels of each biocatalyst. It will be understood by those skilled in the art that enzyme activity and expression levels may vary between preparations and/or batches.

Styrene is produced by converting the L-Phe to trans-cinnamic acid with the first biocatalyst and converting the trans-cinnamic acid to styrene with the second biocatalyst. The producing step is preferably conducted at a pH in a range from 6 to 8. Preferably, the producing step is conducted at a temperature in a range of from 0 to 60° C., more preferably at a temperature in a range from 20 to 40° C., most preferably in a range of from 30 to 37° C. At lower temperatures, for example, in a range of from 0 to 20° C., the reactions will proceed, albeit at a slower rate of reaction. Temperatures above 60° C. may result in styrene polymerization and should therefore be avoided.

A further advantage of the present invention, as compared to conventional approaches using growing cells, is in the recovery of the styrene product. In conventional processes, the styrene is recovered with other growth-related products and by-products. Accordingly, further purification is required. The method of the present invention allows for recovery of substantially pure styrene.

The method of the present invention is readily adaptable to larger-scale production by avoiding issues relating to toxicity and product recovery. Styrene may be recovered in a continuous process drawing an overhead stream containing volatile styrene and using a cold separator to condense the styrene from the overhead stream. Lyophilised whole-cell biocatalysts add a further advantage for large-scale production by providing the whole-cell catalysts in a condition that is easier to handle and store.

### EXAMPLES

The following non-limiting examples of embodiments of the method of the present invention as claimed herein are provided for illustrative purposes only.

### Materials and Reagents

All chemicals, reagents, organic solvents, and other materials and Laboratory consumables used in these examples were of analytical grade and obtained from Merck Life Science UK Limited, Fisher Scientific UK Limited, Agilent Technologies UK Limited, Roche Applied Sciences, and VWR International Limited. Reagents and solvents used for High Performance Liquid Chromatography (HPLC) analysis were of HPLC grade. Standards of L-Phe, trans-cinnamic acid and styrene were of high analytical grade for the preparation of the standard curves. Naturya organic spirulina powder and the KetoChef cold pressed defatted pumpkin seeds protein powder both were purchased from Amazon, UK.

The Escherichia coli (E. coli) strain BL21 (DE3) was from New England BioLabs Inc. The L-Phe producing E. coli strain NST74 (ATCC 31884) was obtained from the American Type Culture Collection (ATCC). The pBbA1C-RFP vector from the BgIBrick series of vectors was obtained from Addgene. CloneAmp HiFiPCR™ premix (Clontech) and the In-Fusion HD Cloning Plus™ kit was obtained from Takara Bio Inc. The Q5® High-Fidelity DNA Polymerase and DpnI from New England BioLabs Inc. and the QIAquick PCR™ purification kit were obtained from Qiagen. Gene sequencing and oligonucleotides syntheses were performed by Eurofins MWG (Ebersberg, Germany).

### Production of First Biocatalyst having Phenylalanine Ammonia-Lyases (PALs)

A recombinant PAL plasmid, RgPAL from Rhodotorula glutinis in a pET16b vector, was obtained. For expression in E. coli cells, the PAL plasmid was transformed into E. Coli BL21 (DE3) strain and grown at 37° C. in LB medium containing 100 μg/mL ampicillin. The cells were grown until the OD₆₀₀ reached approximately 0.6 and induced by adding 0.4 mM IPTG. The cells were incubated overnight at 24° C. at 180 rpm. The cells were harvested by centrifugation for 10 min at 7,000 g at 4° C. The pellets were washed by resuspension three times in phosphate buffer saline (PBS) buffer pH 7.4 followed by centrifugation and removing the liquid buffer.

Resting whole-cell pellets having PAL enzyme was stored in aliquots at −20° C. Lyophilised whole-cell pellets having PAL enzymes were prepared by flash freezing the washed pellets in liquid nitrogen and then subjecting the flash-frozen pellets to freeze drying. The lyophilised cells were stored at 4° C. and at room temperature (25° C.) until use.

### Production of Second Biocatalyst having Fdc1 Enzyme

Co-expression of Aspergillus niger Fdc1 (AnFdc1) with the E. coli UbiX (in E. coli strains lacking T7 polymerase) was achieved by cloning both genes into pBbA1C vector possessing a chloramphenicol resistance marker as described in Messiha et al. (“A Biological Route to Conjugated Alkenes: Microbial Production of Hepta-1,3,5-triene” ACS Syn Biol 10:228-235; 2021).

AnFdc1 was amplified from an existing AnFdc1pET30a construct using CloneAmp™ (Clontech). The template was removed by DpnI (NEB) digest and the DNA was purified using a QIAquick PCR™ purification kit (Qiagen). The gene was cloned into NdeI/BamHI linearized pBbA1C using the In-Fusion HD Cloning Plus™ kit (Clontech) to produce plasmid AnFdc1pBbA1C. Once the plasmid had been sequence verified, E. coli UbiX was amplified from an existing EcUbiXpET21b construct using CloneAmp™ (Clontech). The original template was removed by DpnI and the DNA was purified. The insert was cloned into AnFdc1pBbA1C linearized with BamHI/XhoI using In-Fusion HD Cloning Plus™ kit (Clontech). The resulting AnFdc1/EcUbiX in pBbA1C plasmid allowed co-expression of both genes from a short operon under control of the pTrc promoter.

AnFdc1/EcUbiX was transformed and expressed in E. coli BL21 (DE3) and grown at 30° C. in LB medium supplemented with 17 μg/mL chloramphenicol in a shaking incubator at 180 rpm. The cells were induced with 0.2 mM IPTG and supplemented with 1 mM MnCl₂ at mid log phase (OD_{600 nm˜0.6}) and grown overnight at 25° C./180 rpm. The cells were harvested by centrifugation for 10 min at 7,000 g at 4° C. The pellets were washed by resuspension three times in phosphate buffer saline (PBS) pH 7.4 followed by centrifugation and removing the liquid buffer.

Resting whole-cell pellets having Fdc1 enzyme was stored in aliquots at −20° C. Lyophilised whole-cell pellets having Fdc1 were prepared by flash freezing the washed pellets in liquid nitrogen and then subjecting the flash-frozen pellets to freeze drying. The lyophilised cells were stored at 4° C. and at room temperature (25° C.) until use.

The prepared whole-cell biocatalysts were tested for styrene production from different sources for L-Phe. Casamino acids were used as a source of free L-Phe. Blue green spirulina algae and pumpkin seed powders were hydrolysed to extract L-Phe. Finally, L-Phe was produced by fermentation.

### Styrene Production from Protein-Rich Feedstock

Styrene production was tested using 20g cdw/L of resting whole-cell biocatalysts having RgPAL or AnFDC1/EcUBiX, 50 mM sodium phosphate buffer pH 7.0, 30° C. at 180 rpm with L-Phe sources, including casamino acids, blue green algae spirulina powder, and defatted pumpkin seeds powder. For the spirulina powder and the defatted pumpkin seeds powder, an acid hydrolysis step with 6N HCl was performed (50 ml acid for every g) under reflux for 24 hr to hydrolyse the proteins into amino acids followed by a neutralization step with NaOH to pH 7.0. Casamino acids powder (1 g) was dissolved in water to achieve 0.1 g/mL dissolved in water.

The reactions were carried out in tightly sealed vials for at least 4 hours and in some cases for overnight to monitor any changes in the product profile.

L-Phe, trans-cinnamic acid and styrene were analysed by HPLC. Samples from the biotransformation reactions were prepared by adding acetonitrile (HPLC grade) to stop the reaction at volume ratio of 1:1 and centrifuged at 14,000 rpm for 10 min. Supernatants were processed through 0.22-micron filters and transferred to glass HPLC vials and sealed with a Teflon-lined cap. Twenty microliters (unless otherwise stated) of each sample were injected (unless otherwise stated). The reverse phase HPLC analysis was carried out on an Agilent 1260 Infinity II LC™ system. An Eclipse XDB-C18™ column (5 μm, 4.6×150 mm) from Agilent was used. The column was eluted with Solvent A (HPLC grade water with 0.1% trifluoroacetic acid, TFA) and Solvent B (HPLC grade acetonitrile with 0.1% TFA) at a total constant flow rate of 1.0 ml/min. The eluent was kept for first 2 min as a mixture of 80% Solvent A and 20% Solvent B, then followed by linear gradient elution with 20% Solvent A and 80% Solvent B for 6 min, and finally held constant with a mixture of 80% Solvent A and 20% Solvent B for 3 min. The column temperature was maintained at 35° C. DAD detector was set to collect data at 254 nm, 273 nm, and 245 nm. Standard curves for L-Phe, trans-cinnamic acid and styrene were prepared for quantitative analysis.

All reactions were performed in biological and analytical replicates. At least four preparations of each biocatalyst were tested during 6 months of storage and were shown to be stable.

### Casamino Acids

Production of styrene from casamino acids was achieved in 2 hours by adding 20 mg resting cells of RgPAL and AnFdc1/EcUbiX in 1 ml reaction volumes (contains 0.1 g casamino acids/mL). The L-Phe content in the reaction was 26.9±1.8 mM and the produced styrene was 22.08±0.01 mM (a titre of 2.3 mg/ml) in the reaction after 2 hrs, accounting for 82.2% of the expected titre for full conversion of L-Phe to styrene. This confirms the biotransformation is not inhibited by other amino acids.

### Blue Green Spirulina Algae & Pumpkin Seed Powders

The whole-cell biocatalysts were then tested using blue green spirulina algae and pumpkin seed powders (containing approximately 65-70% and 60-65% protein content respectively) as model sources of L-Phe after acid hydrolysis treatment and neutralisation. Following the addition of the biocatalysts (20 mg/mL) in 1 mL reaction volumes that contained the hydrolysed spirulina powder or pumpkin seeds powder, styrene was produced with a titre of 0.93 mg/mL in case of spirulina and 0.14 mg/mL in case of pumpkin seeds powder in 2 hours. For Spirulina powder, L-Phe content in the biotransformation reaction was 9.6±0.8 mM, the styrene product was 8.9±0.6 mM after 2 hrs, accounting for 93% of the expected titre. In case of pumpkin seeds powder, L-Phe content in the reaction was 1.6±0.2 mM and the produced styrene was 1.3±0.2 mM after 2 hrs, accounting for 83% of the expected titre (considering the dilution factors due to hydrolysis and neutralization).

The results reveals that the developed protocol could be widely applied to produce styrene from various L-Phe containing sources and is not inhibited by other biomass components.

### Production of L-Phe by Fermentation

The L-Phe producing strain E. coli NST74 (ATCC 31884) was employed for fermentative production of L-Phe. Cultures were initially grown in batch fermentation in MM12 media at 33° C. in a small laboratory bioreactor (330 mL volume) for 48 hrs where the pH was controlled at 7.0 by an alkaline mixture consists of 0.5 M NaOH, 0.5 M KOH and 0.35 M NH₄OH. Cell growth, pH and temperature were monitored, and L-Phe production was quantified by HPLC.

Table 1 summarizes the production of L-Phe from E. coli NST74 grown in MM12 media at 33° C. in batch fermentation for 38 hours.

TABLE 1

| | |
|---|---|
| Time | L-Phe Production |

| (hr) | (mM) | (g/L) |
|---|---|---|
| 0 | 0 | 0 |
| 14.5 | 0.36 | 0.06 |
| 19.5 | 1.0 | 0.17 |
| 21.7 | 2.5 | 0.42 |
| 24 | 4.2 | 0.69 |
| 38 | 10.7 | 1.8 |

### Styrene Production from L-Phe using Whole-Cell Biocatalysts

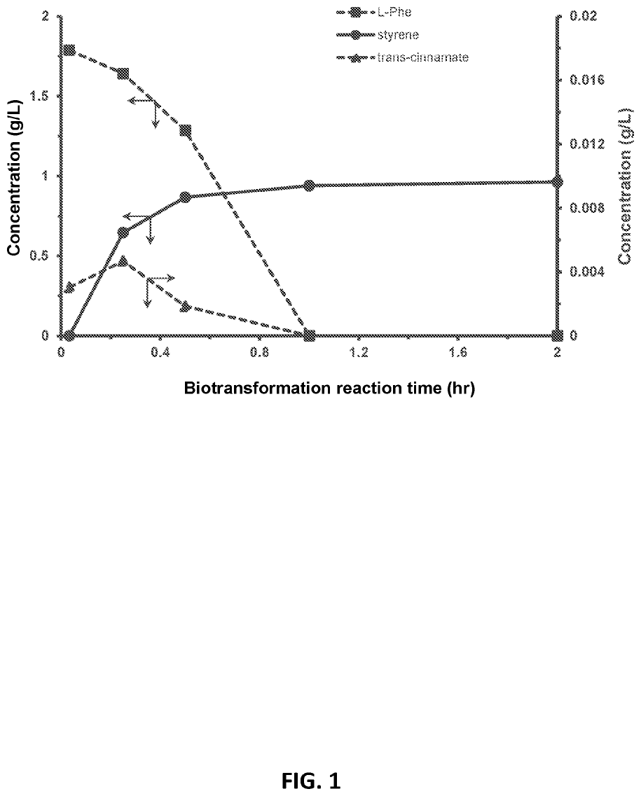

After fermentation was complete, the whole-cell biocatalysts having Fdc1 and PAL enzymes were added to the culture broth. Styrene production was performed using 20 g cdw/L of each whole-cell biocatalyst in a resting phase or 5 g/L of each whole-cell biocatalyst in the lyophilised condition, 50 mM sodium phosphate buffer pH 7.0, 30° C. or 33° C., 180 rpm in 7-mL sealed vials. The reactions were carried out for 2-4 hours and in some cases for overnight. The results for lyophilised whole-cells after 2 hours are shown in FIG. 1. Results for resting whole-cells were substantially the same.

Table 2 presents a summary of the styrene titres achieved by using lyophilised whole-cells (5 g/l) of each of the RgPAL and AnFdc1EcUbiX biocatalysts with concentrations of up to 264.9 mM L-Phe (43.76 g/l). Meanwhile, Table 3 presents a summary of the styrene titres achieved by using resting whole-cells (20 g cdw/L) of each of the RgPAL and AnFdc/EcUbiX biocatalysts with concentrations of up to 104.2 mM L-Phe (17.21 g/l). L-Phe was added at the desired concentrations. However, as shown in Tables 2 and 3, after adding the whole-cell biocatalysts, the L-Phe content was analysed to determine the actual concentration at 0 hr. The whole-cell biocatalysts contain cellular levels of L-Phe that contributes in part to the total L-Phe content in the biotransformation reaction.

Tables 2 and 3 illustrate results for reaction of 5 g/L of lyophilised and 20g cdw/L resting whole-cells, respectively, of RgPAL and 5 g/L and 20g cdw/L resting whole-cells, respectively, of AnFdc1/EcUbiX in 50 mM sodium phosphate buffer pH 7.0, 30° C. at 180 rpm in 1 mL reaction volumes, unless otherwise stated.

Complete consumption of L-Phe was achieved within 2-4 h, and in most cases in less than 2 h, depending on the biocatalyst batch preparations and the concentration of L-Phe. Styrene product levels were found to be to be about 88.1-97.4% (91.7±2.5%) of the expected titres. The percentage styrene values were calculated as % of the expected titre if full consumption of L-Phe was observed, assuming no loss of styrene occurs during sample preparation, dilution, and analysis. Some styrene was lost during sample preparation, dilution and analysis owing to its volatile and inherently reactive nature.

As shown by Tables 2 and 3, substantially the same results were found using 20 g cdw/l resting whole-cells and 5 g/L lyophilised whole-cells of each of the RgPAL and AnFdc1/EcUbiX biocatalysts. This shows that the biocatalysts were not deleteriously affected by the lyophilisation process.

Full conversion of L-Phe to styrene with no evidence of trans-cinnamate accumulation was obtained with L-Phe concentrations up to 100 mM. For higher L-Phe concentrations (>100 mM), a small amount of trans-cinnamate (1-2.5 mM) was occasionally observed; accounting for about 1-2.5% of the L-Phe concentration under the conditions used only when the reaction time exceeds 12 hrs. This suggests that at higher styrene and CO₂ concentrations accumulated under the closed volume reaction conditions used, the Fdc1 mediated reaction does not go to completion.

It should be noted that the solubility of L-Phe does not extend much beyond 100 mM at room temperature (25° C.), but higher styrene titres were obtained when (super) saturated L-Phe solutions or L-Phe suspensions were used in the biotransformation reactions at 30° C. This yielded a production of up to 18.3 g/L styrene and demonstrates L-Phe availability as one of the main limiting factors, offering a promising green route towards industrial bioproduction of styrene. To further improve this strategy for industrial application, the styrene product was captured from the biotransformation reactions (at 37° C.) by use of a cold trap for one test in Table 2, as noted therein.

TABLE 2

| L-Phe substrate | |
|---|---|
| concentration^{[a]} | Styrene product |

| mM | mM | g/L | %^{[b]} |
|---|---|---|---|
| 11.6 ± 0.2 | 10.9 ± 0.2 | 1.13 ± 0.02 | 93.6 ± 1.7 |
| 22.7 ± 0.2 | 20.0 ± 0.9 | 2.08 ± 0.09 | 88.1 ± 4.0 |
| 41.9 ± 0.4 | 37.2 ± 2.3 | 3.88 ± 0.24 | 88.9 ± 5.4 |
| 64.1 ± 0.1 | 58.3 ± 2.2 | 6.10 ± 0.23 | 90.9 ± 3.4 |
| 81.3 ± 0.3 | 75.9 ± 2.5 | 7.90 ± 0.26 | 93.4 ± 3.1 |
| 100.5 ± 0.1 | 92.1 ± 0.3 | 9.59 ± 0.03 | 91.6 ± 0.3 |
| 178.5 ± 5.8^{[c]} | 157.5 ± 10 | 16.4 ± 1.1 | 88.2 ± 5.6 |
| 264.9 ± 5.2^{[c][d]} | 230 ± 11 | 23.9 ± 1.1 | 91.6 ± 4.3 |

^{[a]}The measured concentrations of L-Phe in the reaction at 0 hr.
^{[b]}Styrene product calculated as % of the expected titre if full production is achieved (full consumption of L-Phe as observed, assuming no loss of styrene occurs during sample preparation, dilution, and analysis).
^{[c]}(Super)saturated solutions of L-Phe
^{[d]}Styrene product captured by a cold trap, calculated as % of the expected titre (250.9 mM) based on the calculated uncaptured styrene, unreacted L-Phe and accumulated trans-cinnamate in the biotransformation reaction after 4 hrs. Reaction conditions: supersaturated L-Phe at 37° C., 1 g of lyophilised RgPAL and AnFdc1/EcUbiX in 50 mM sodium phosphate buffer pH 7.0, 100 rpm at 37° C. in 100 mL reaction volumes

TABLE 3

| L-Phe substrate | | | |
|---|---|---|---|
| concentration | Styrene product | | |
| mM | mM | g/L | % |
| 11.8 ± 0.3 | 11.5 ± 0.2 | 1.2 ± 0.01 | 97.4 ± 1.6 |
| 21.9 ± 0.1 | 20.1 ± 0.7 | 2.09 ± 0.07 | 91.7 ± 3.1 |
| 41.8 ± 0.2 | 37.6 ± 1.8 | 3.92 ± 0.19 | 89.9 ± 4.3 |
| 62.8 ± 0.3 | 57.8 ± 1.9 | 6.02 ± 0.2 | 92.0 ± 3.0 |
| 82.4 ± 0.5 | 77.6 ± 0.7 | 8.08 ± 0.07 | 94.2 ± 0.8 |
| 104.2 ± 0.5 | 96.3 ± 2.1 | 10.03 ± 0.22 | 92.4 ± 2.0 |

[a] The measured concentrations of L-Phe in the reaction at 0 hr,
[b] Styrene product calculated as % of the expected titre if full production is achieved (full consumption of L-Phe as observed, assuming no loss of styrene occurs during sample preparation, dilution, and analysis).

For the test at 264.9 mM L-Phe, styrene product was captured from the biotransformation reactions by cold trapping inside a fume hood. The biotransformation reactions were either carried out in flasks or in a controlled chemical reactor attached to a customized cold trap system and the styrene product was collected in cold acetonitrile at −20° C. Reactions carried out in flasks do not reach completion due to an increase in pH (uncontrolled pH beyond the buffer capacity) therefore reactions (100 mL volumes) were carried out in an EasyMax 102™, METTLER TOLEDO chemical reactor that contains the desired concentration of L-Phe, 200 μl of 10% antifoam 204 and 1g of each lyophilised biocatalyst in 50 mM sodium phosphate buffer pH 7.0 for 4 hrs at 100 rpm. The temperature was controlled at 37° C. and the pH was maintained at pH 7.0 by the addition of 3 M HCl. An air pump was connected to the biotransformation reaction in the chemical reactor to purge air onto the bioreactor vessel and the styrene product was passed on dried CaCl₂ to and then captured by the cold trap inside a fume hood. The cold trap containing acetonitrile (100 mL) was immersed in cold isopropanol that was cooled to −20° C. by a Julaba FT902T™ immersion cooler. Up to 23.9±1.1 g/L (230±11 mM) highly pure styrene was captured from L-Phe (264.9±5.2 mM) in 4 hrs.

Without being bound by theory, the improvement in styrene titre over earlier published studies is due to a combination of several factors, including the co-expression of EcUbiX (yielding active Fdc1), overcoming the toxicity of styrene on living cells (by using resting E. coli cells), use of higher L-Phe concentrations in the biotransformation reactions, maintaining the pH over the reaction time and the continuous capture of styrene from the reaction.

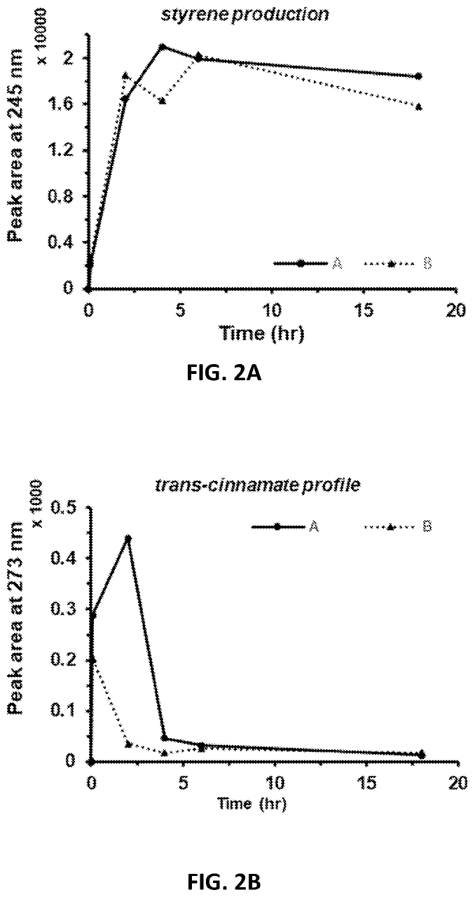

FIGS. 2A and 2B compare styrene production and trans-cinnamate production/consumption profiles for freshly prepared lyophilised whole-cell biocatalysts (A) and lyophilised whole-cell biocatalysts after 9 months storage at room temperature (B). The tests were conducted using 10 mg/mL RgPAL and 10 mg/mL AnFdc1/EcUbiX starting with 100 mM L-Phe. The data demonstrates good stability of the lyophilised whole-cell biocatalysts after 9 months of storage at room temperature.

The efficiency of the biotransformation protocol was identical when using various quantities of lyophilised cell biocatalysts (1-20 g/l), as well as resting cells (5-100 cdw/l). The lyophilised biocatalysts were employed at those levels as the lyophilised cell weight is approximately 20-25% of the corresponding resting cell weight. Quantities up to 20 g/L were initially selected to test the performance and stability of the lyophilised biocatalysts.

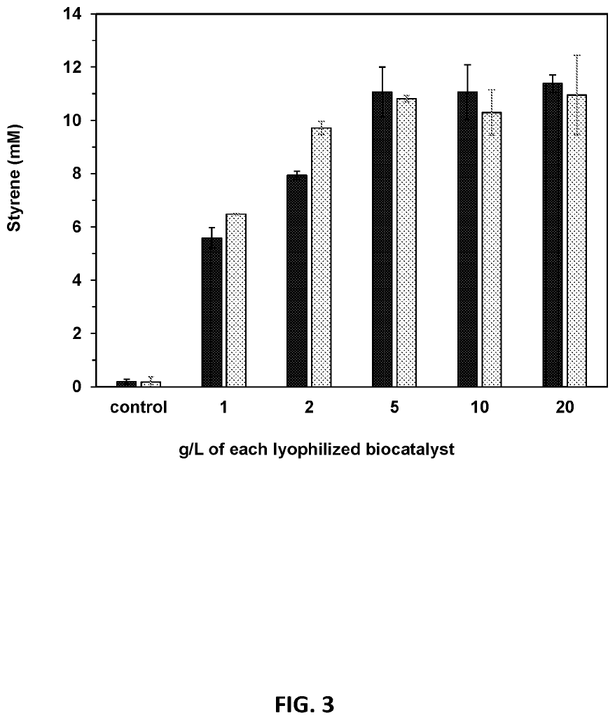

The results shown in FIG. 3 demonstrate that employing modest quantities of lyophilised biocatalysts (as small as 1 g per litre reaction) are able to produce styrene from L-Phe under the conditions used. FIG. 3 shows the results for styrene production using lyophilised biocatalysts (0-20 g/L of each biocatalyst) in the biotransformation reaction. In each pair of columns, the first column represents average replicate measurements after 4 hours of rection time ±standard deviation, while the second column represents average replicate measurements after 16 hours reaction time ±standard deviation.

While the embodiments are described with reference to various implementations and exploitations, it will be understood that these embodiments are illustrative and that the scope of the inventive subject matter is not limited to them. Many variations, modifications, additions, and improvements are possible.

1. A process for producing styrene, the process comprising the steps of:
providing a source of L-phenylalanine to a vessel;
adding a first biocatalyst comprising a PAL enzyme to the vessel, the first biocatalyst provided in a form selected from PAL-containing whole-cell pellets, a suspension of PAL-containing whole-cell pellets, a suspension of PAL-containing whole cells derived from PAL-containing whole-cell pellets, and combinations thereof;
adding a second biocatalyst comprising a Fdc1 enzyme to the vessel, the second biocatalyst provided in a form selected from Fdc1-containing whole-cell pellets, a suspension of Fdc1-containing whole-cell pellets, a suspension of Fdc1-containing whole cells derived from whole-cell pellets, and combinations thereof; and
producing styrene by converting the L-phenylalanine to trans-cinnamic acid with the first biocatalyst and converting the trans-cinnamic acid to styrene with the second biocatalyst.

2. The process of claim 1, wherein the PAL-containing whole-cell pellets are lyophilised whole-cell pellets.

3. The process of claim 1, wherein the PAL-containing whole-cell pellets are resting whole-cell pellets.

4. The process of claim 1, wherein the Fdc1-containing whole-cell pellets are lyophilised whole-cell pellets.

5. The process of claim 1, wherein the Fdc1-containing whole-cell pellets are resting whole-cell pellets.

6. The process of claim 1, wherein the source of L-phenylalanine is prepared by fermenting a renewable feedstock.

7. The process of claim 6, wherein the renewable feedstock is selected from the group consisting of saccharides, alcohols, organic acids, fatty acids, and combinations thereof.

8. The process of claim 6, further comprising the steps of fermenting the renewable feedstock in the presence of a microorganism capable of producing L-phenylalanine, allowing the fermenting step to proceed to predetermined level of L-phenylalanine in a fermentation broth, wherein the first whole-cell pellet and the second whole-cell pellet are added to the fermentation broth after the predetermined level of L-phenylalanine is achieved.

9. The process of claim 1, wherein the source of L-phenylalanine is prepared by extraction from a protein-rich renewable feedstock.

10. The process of claim 9, wherein the protein-rich renewable feedstock is selected from the group consisting of dried distillers grain, algal oil, wastes from bioethanol, bio-oil and food production, plant leaves, poultry feather meal, and combinations thereof.

11. The process of claim 9, wherein the extraction is selected from acid hydrolysis, enzymatic hydrolysis, amino acid fractionation, and combinations thereof.

12. The process of claim 1, wherein the source of L-phenylalanine is a feed rich in L-phenylalanine selected from the group consisting of casamino acid, pumpkin seeds, algae, and combinations thereof.
